(19) United States
(12) Patent Application Publication
(10) Pub. No.: US 2012/0150247 A1
(43) Pub. Date: Jun. 14, 2012

(54) Battery pack topology

(76) Inventors: Giovanni C. Meier, Madison, CT (US); Gintaras A. Vaisnys, Chicago, IL (US)

(21) Appl. No.: 12/928,320

(22) Filed: Dec. 8, 2010

(51) Int. Cl. A61N 1/39; H02J 7/00

(52) U.S. Cl. 607/5; 320/126

(57) ABSTRACT

A battery pack topology wherein the battery pack has multiple battery sub-stacks electrically connected in parallel such that the capacity of each battery sub-stack may be utilized but one is reduced unequally as to the others. As a result, one battery sub-stack will reach a point of failure before the other, which causes a drastic, observable change in the output voltage of the battery pack, but provides sufficient reserve capacity to permit a user of a device, such as an AED, having the battery pack to be notified in a timely fashion of the need to replace the battery pack.

### TECHNICAL FIELD

The present invention relates to automated external defibrillators, and, more specifically, to a battery pack for powering the device.

### BACKGROUND OF THE INVENTION

External defibrillators are emergency medical devices designed to supply a controlled electric shock (i.e., therapy) to a person's (e.g., victim's) heart during cardiac arrest. This electric shock is delivered via pads that are electrically connected with the external defibrillator and in contact with the person's body.

To provide a timelier rescue attempt for a person experiencing cardiac arrest, some external defibrillators have been made portable, by utilizing battery power (or other self-contained power supplies). In addition, many portable external defibrillators have programming to make medical decisions making possible operation by non-medical personnel.

These portable external defibrillators, commonly known as automated external defibrillators (AEDs), including automatic and semi-automatic types, have gained acceptance by those outside the medical profession and have been deployed in myriad locations outside of traditional medical settings. Due to the life saving benefits of AEDs, more and more non-medical users are purchasing and deploying AEDs in their respective environments. This allows for a rescue attempt without the delay associated with bringing the person to a medical facility, or bringing a medical facility to the person (e.g., a life support ambulance).

Individuals as well as businesses are purchasing and deploying AEDs. As time is of the essence during any rescue attempt, multiple AEDs may be purchased by any particular individual or user to allow placement at multiple locations. In the case of an individual, this could be on several floors of a home, and in the case of a business, this could be for placement throughout a facility (e.g., factory, office building, or large retail center). Thus, regardless of where the victim is within the home/facility, access to an AED would only be seconds, or minutes, away.

AEDs rely on batteries to provide power. More precisely, AEDs rely on battery packs that have battery stacks, which contain multiple batteries (i.e., cells). To assure that the battery pack is capable of meeting the power demands of the AED, the capacity of the battery pack is continually assessed.

Generally, assessment of the present capacity of the battery pack occurs during routine AED self testing (e.g., schedule, autonomous testing conducted by the unit). If an assessment determines that the battery pack lacks sufficient capacity to perform to a predetermined level, the user is alerted to the need to replace the battery pack.

When to alert a user as to the need to replace the battery pack can be extremely problematic. If a user is alerted too early, battery pack capacity is wasted, as the user replaces a battery pack that could perform. If a user is alerted to late, the AED could be out of service before the timely replacement of the battery pack can occur.

Determining when to alert a user to replace a battery pack is complex. Typically, battery pack capacity is assessed by determining the voltage output delivered under specific load conditions, which places a known load on the battery such that the battery's internal resistance causes a decrease in voltage output. If the voltage output falls below a given pre-determined threshold voltage, the battery pack is considered to lack the necessary capacity. In other words, voltage output is a surrogate for remaining battery pack capacity, thus remaining battery pack life.

Historically, batteries, and the battery packs that use them, had a discharge curve that exhibited a gradual voltage output decline under load. Thus, a threshold voltage output under a known load of a battery pack could be identified that equated to battery pack end of life.

As battery technology has advanced, the discharge curve has flattened out, thus the gradual output voltage decline has been eliminated. More precisely, newer technology batteries, such as Lithium Battery CR-2/3A, exhibit relatively stable voltage output under a known load until near end of life when there is a precipitous drop.

Presently, to provide a timely warning to an AED user of the need to replace a battery pack using newer technology batteries, the threshold voltage under a known load is being continually increased. However, as the threshold voltage under a known load is increased, due to the ever flatter discharge curves, it is becoming ever closer to the normal operating output voltage. As those skilled in the art of assessing remaining battery capacity will appreciate, as the threshold voltage output under load approaches the normal operating output voltage under load, it becomes increasing difficult, due to the ever smaller delta between the two and minor fluctuations in the output voltage due to manufacturing and operational tolerances, to discern when the threshold voltage output has been reached. As a result, to meet the need of assuring proper operation and a timely notification of users as to the need to replace the battery, users are being instructed to replace battery packs earlier than might otherwise be required. As a result, capacity in battery packs employing newer technology batteries is being wasted.

What is needed in the art is a better method of assessing battery pack end of life so additional battery capacity can be utilized to lower user costs. More specifically, autonomous self-tests being conducted on the AED should be able to determine the remaining capacity of the battery pack. Then, the battery pack should remain fully functional for some reasonable period of time thereafter to permit the timely notification of a user as to the need to replace the battery pack and allow a reasonable time to allow replacement before the battery pack is depleted.

Furthermore, other desirable features and characteristics of the present invention will become apparent for the subsequent detailed description of the invention and the appended claims, taken in conjunction with the accompanying drawings and this background of the invention.

### SUMMARY OF THE INVENTION

The invention is a battery pack topology wherein the battery pack has multiple battery sub-stacks electrically connected in parallel such that the capacity of each battery sub-stack may be utilized but one is reduced unequally as to the others. As a result, one battery sub-stack will reach a point of failure before the other, which causes a drastic, observable change in the output voltage of the battery pack, but provides sufficient reserve capacity to permit a user of a device, such as an AED, having the battery pack to be notified in a timely fashion of the need to replace the battery pack.

In an exemplary embodiment, the battery pack includes two battery stacks configured in parallel. As a result, each battery stack is a battery sub-stack within the battery pack. The inequality in capacity utilization between the battery sub-stacks results from a difference in voltage drop relative to each branch of the parallel circuitry. In an illustrative example, this voltage drop difference is created by employing a different number of diodes on each branch. As those skilled in the art will appreciate, other electronic devices could be used to create different voltage drops, but diodes work well as the voltage drop, which is generally constant, as it is generally independent of current being drawn, except at very low current draws, from the associated battery sub-stack.

Other features, attainments, and advantages will become apparent to those skilled in the art upon a reading of the description when taken in conjunction with the accompanying drawings.

### BRIEF DESCRIPTION OF THE DRAWINGS

FIG. 1 is a top view of an illustrative AED on which the present invention may be used.

FIG. 2 is a perspective side view of the AED depicted in FIG. 1.

FIG. 3 is a functional block diagram of the components of the AED depicted in FIGS. 1 and 2.

FIG. 4 is a block diagram of the battery stack found in the in battery pack.

FIG. 5 is a chart showing battery pack voltage over time. The chart depicts the results of two separate tests. One line is for one test and the other is for a second test. The overlapping of the lines indicates the repeatability of the outcome.

FIG. 6 is a chart showing current sharing between the battery sub-stacks in the battery pack where the current draw is 1 milliamp.

FIG. 7 is a chart showing current sharing between the battery sub-stacks in the battery pack where the current draw is 100 milliamps.

FIG. 8 is a chart showing current sharing between the battery sub-stacks in the battery pack where the current draw is 1.5 amps.

FIG. 9 is a schematic drawing of an alternate load allocator.

FIG. 10 is a schematic drawing of another alternate load allocator.

### DETAILED DESCRIPTION OF EXEMPLARY EMBODIMENTS

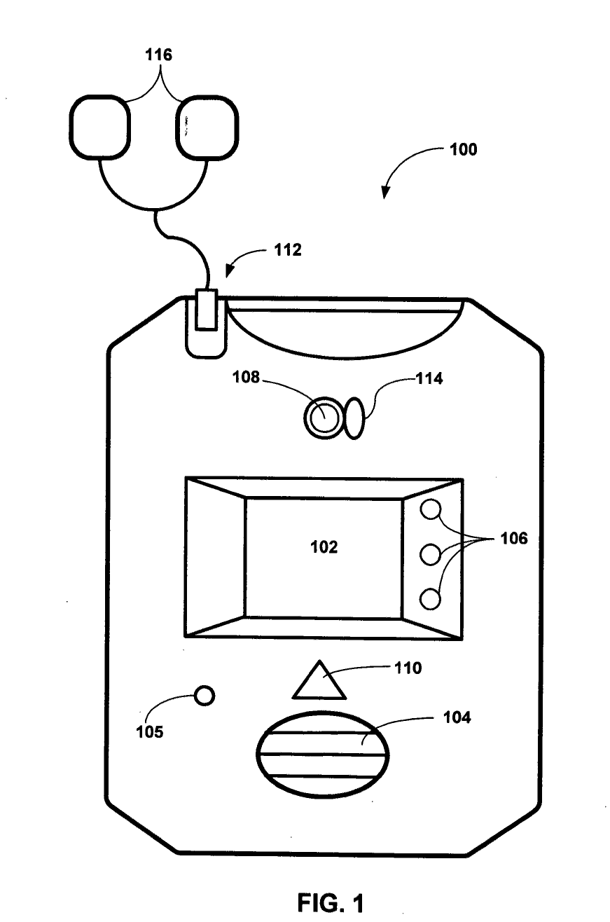

Turning now to the drawings, FIG. 1 illustrates a plan view of an AED unit 100. As seen in this FIG. 1, the AED unit 100 has a video display 102, a speaker 104, an audio output jack 105, and a user interface 106. The AED unit 100 further includes an ON/OFF switch 108, a shock switch 110, a pad connector 112, and an active status indicator 114 (ASI) (e.g., a light source which blinks green indicating the unit is OFF but ready to operate normally, solid green indicating the unit is ON and operating normally, solid red indicating the unit is ON but having a problem, and blinking red indicating the unit is OFF but having a problem. If the ASI is not blinking, the unit is out of service). The pad connector 112 connects pads 116 to the AED unit 100.

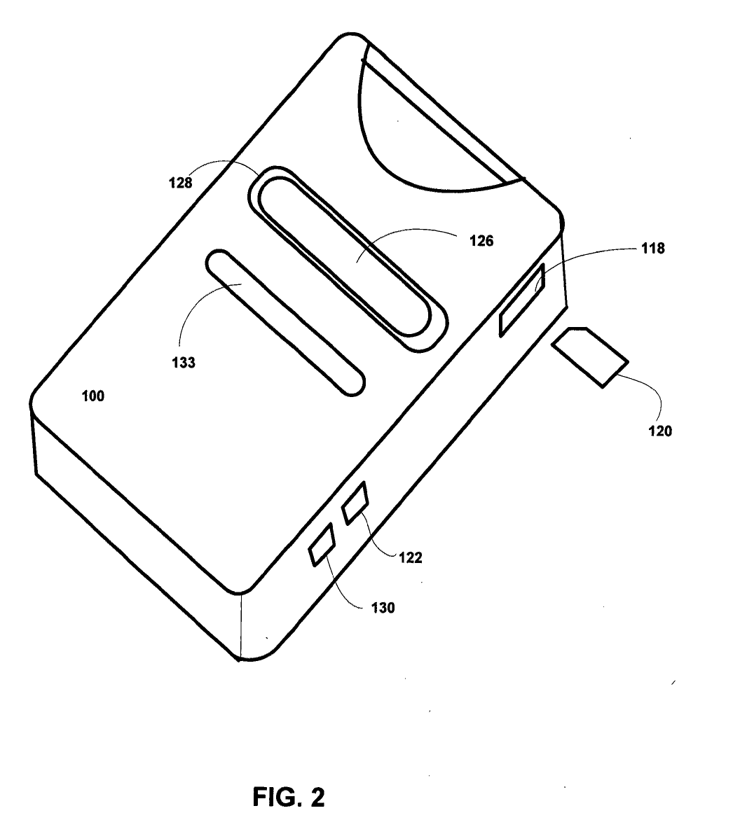

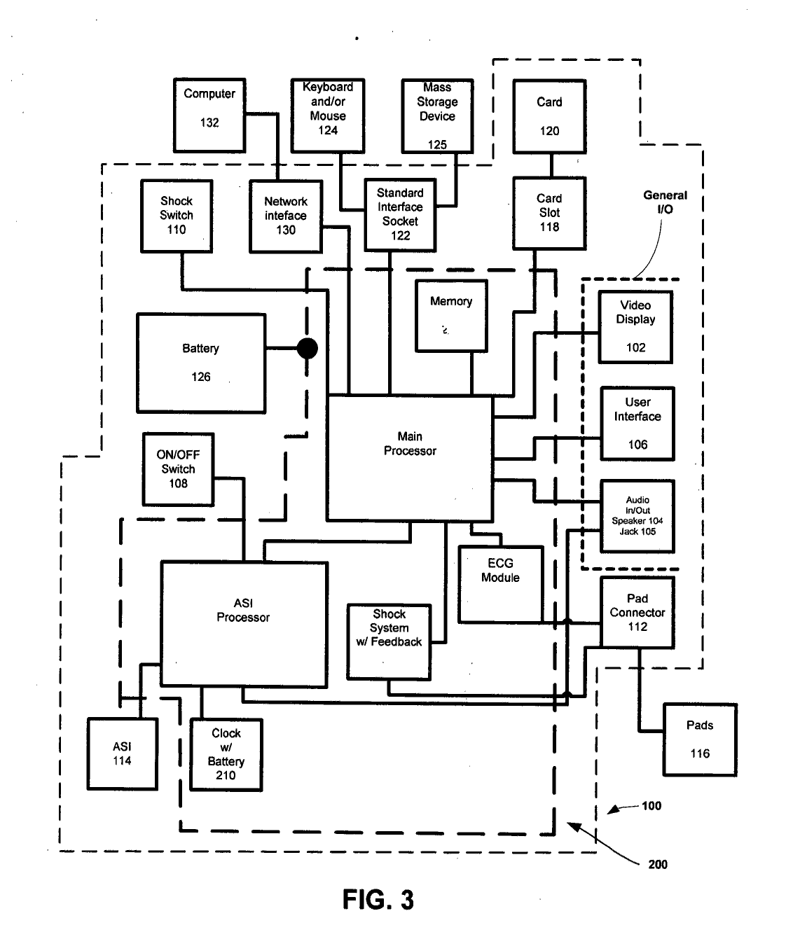

Referring to FIG. 2, the AED unit 100 further includes a card port 118 for providing an electronic interface for a card 120 for data collection, a standardized interface socket 122, e.g., and universal serial bus (more commonly known as a USB port) for connecting such items as a keyboard and/or mouse 124 or a mass storage device 125 (see FIG. 3), and a network interface 130 for connecting, for example a computer 132 (see FIG. 3). Further, the AED unit 100 has a pad slot 133 for securing pads 116.

The AED unit 100 includes a battery pack 126 that provides the main power. As illustrated, the battery pack 126 slides into a battery slot 128, but it could an internal battery pack. Where the battery 126 is removably secured in the battery slot 128, a faulty battery can generally be replaced by a user.

FIG. 3 is a functional block diagram of an exemplary AED unit 100. Circuitry and programming of AED units is well known in the art.

The AED unit 100 typically have many operating modes, with some being sub-modes of primary modes. There are two primary modes—OFF and ON. The OFF mode has several sub-modes including SELF-TEST and AUXILIARY. The OFF-SELF-TEST sub-mode is the default mode. More specifically, the AED unit 100 must always be in an operational mode. Thus, when the AED unit 100 is referred to as being in the OFF mode, it is in one of the sub-modes. When the AED unit 100 is in the OFF SELF-TEST sub-mode, a user considers the AED unit 100 to be OFF.

In the OFF SELF-TEST sub-mode, the circuitry 200 of the AED unit 100 utilizes minimal power to maintain basic functions of the AED such as running a clock 210 (which is shown as having a backup battery) and autonomously (i.e., without human intervention) initiating self-tests, so that scheduled self-diagnostic maintenance checks in response to the passage of time are performed. The results of the self-test in this illustrative AED 100 are displayed by an active status indicator 114, over which the AED programming has autonomous control.

For a rescue attempt, the AED unit 100 is put into the ON mode from the OFF SELF-TEST sub-mode by operation of the ON/OFF switch 108. After the rescue attempt, the AED unit 100 may be put back into the OFF SELF-TEST sub-mode by operation of the ON/OFF switch 108, or the programming may automatically put the AED into the OFF SELF-TEST sub-mode.

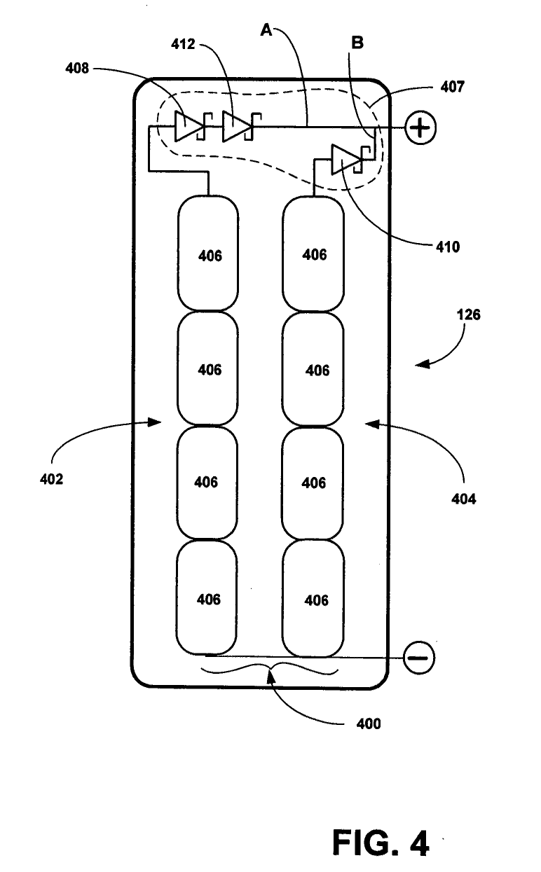

Continuing with FIG. 4, FIG. 4 is a block diagram of the topology of a battery stack (generally referred to by reference no. 400) inside the battery pack 126 (See FIG. 2). Each battery sub-stack 402, 404 is composed of some number of battery cells 406. Each battery sub-stack 402, 404 has an initial capacity sufficient to meet the energy needs of the AED 100 for some period of time beyond a single use.

For a typical AED application, a suitable battery cell 406 is a 3 v battery, such as a Duracell Lithium CR-2/3A, and a battery sub-stack 402, 404 is four batteries electrically connected in series giving the battery sub-stack an initial output voltage of 12 v. These batteries have an initial capacity of about 1.5 Ah. In this exemplary embodiment, each battery sub-stack 402, 404 is generally identical (to the degree permitted by manufacturing tolerances) as they employ the same type and number of battery cells 406, but this is not required.

The two battery sub-stacks 402, 404 are connected via a load allocator 407 that places the battery sub-stacks in parallel. Therefore, one battery sub-stack is on branch A, and the other on branch B.

The illustrated load allocator 407 includes three identical diodes 408, 410, 412 wherein two 408, 412 are in series and in parallel with one 410. The diode configuration of the load allocator 407 (two on branch A and one on branch B) creates an unequal voltage drop across the branches A, B of the battery pack 126. Since the branch voltage drops are unequal, the current drawn over time, or the capacity used, from each individual battery sub-stack 402, 404 will be different. As a result of the load imbalance, battery sub-stack 404 (the battery sub-stack on the one diode branch) will be depleted prior to battery sub-stack 402.

In addition, a diode on each branch of the parallel circuit prevents one battery sub-stack 402, 404 from charging the other battery sub-stack in the event they should have different voltage potentials. As those skilled in the art will appreciate, the identified suitable batteries are not rechargeable; therefore, these batteries should not be subjected to a charging current.

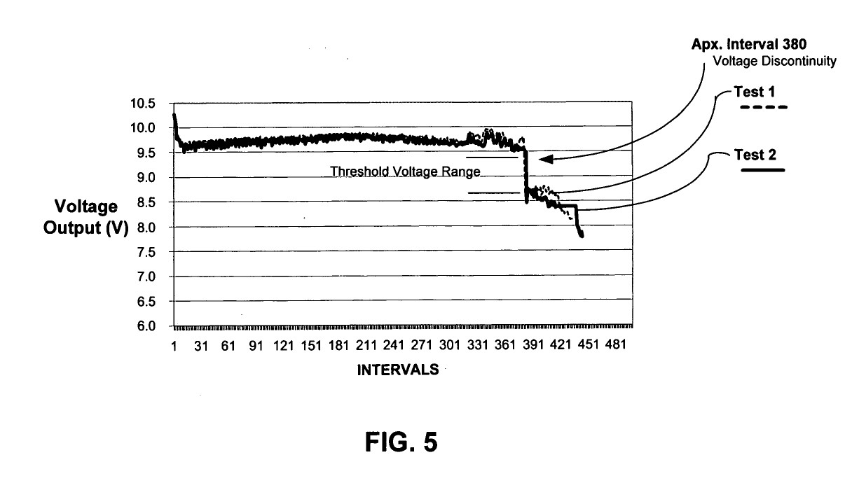

As shown in FIG. 5, the voltage output from the battery pack 126, comprising Duracell Lithium CR-2/3A batteries and using identical Schottky diodes, provides a clear indication of when the battery pack should be replaced.

More precisely, FIG. 5 shows changes in the output voltage of a battery pack 126 undergoing accelerated life testing. The accelerated life test simulates an AED “battery test event” (e.g., a draw at approximately 2 amps for 2 seconds) at fixed intervals.

As shown in FIG. 5, the battery pack 126 has an initial steady voltage output of approximately 10.25 volts under load. After a few simulated intervals, the voltage output drops to approximately 9.75 volts under load, which is generally maintained until approximately simulated interval 380. After approximately simulated interval 380, a significant drop, or discontinuity, in voltage output under load is observed. After the discontinuity at simulated interval 380, the voltage output dropped to roughly 8.6 volts under load.

As used herein, a voltage discontinuity means a precipitous voltage output drop of the battery pack from one operational voltage to another under a known load. An operational voltage means a voltage in combination with a remaining capacity that is capable of operating the device for at least one cycle.

The voltage output discontinuity results from the failure of the ability of one battery sub-stack to provide any current. In other words, prior to the failure of one battery sub-stack, both battery sub-stacks contributed current and the resulting output voltage was 9.75 volts under load. After the voltage discontinuity, which resulted from an end of life event wherein one battery sub-stack (e.g., a failure of at least one battery cell 406 in the battery sub-stack 404), the current draw on the remaining battery sub-stack resulted in a voltage output under load of 8.6 volts. The testing was continued until a simulated interval 420, where at that point the battery pack 126 was unable to provide an operational voltage.

This accelerated life test indicates the battery pack 126 had sufficient operational voltage to operate for a simulated 420 intervals and give a noticeable event at approximately simulated interval 380. This noticeable event, of output voltage discontinuity, can be used to alert a user of a need to replace the battery, which is discussed below.

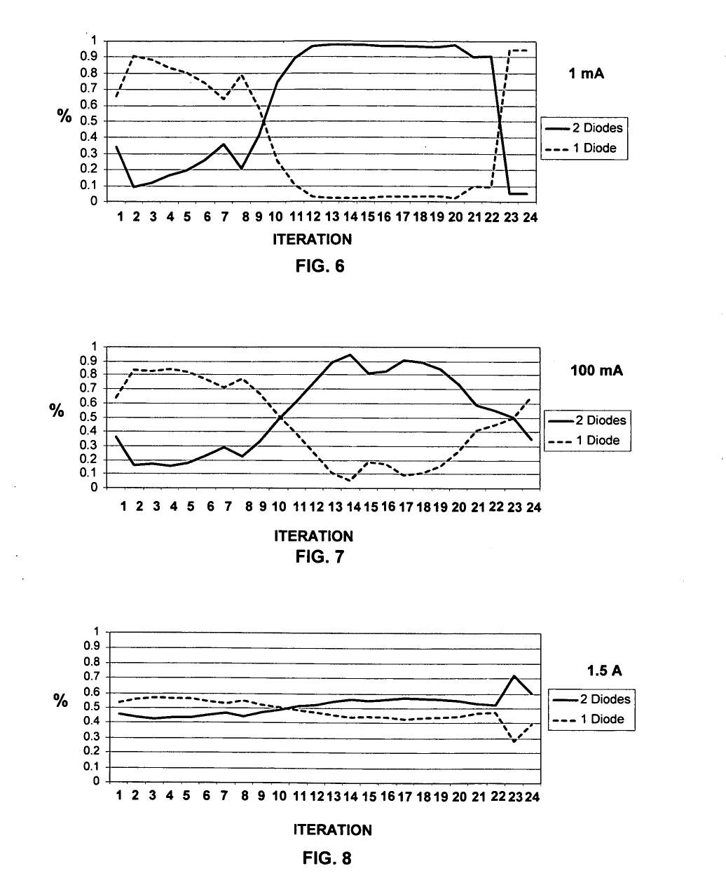

The different capacity being drawn from each battery sub-stack, or load sharing between the battery sub-stacks, under different load conditions is shown in FIGS. 6-8. Each battery sub-stack has a total capacity, or amp-hrs. When the AED is in an operational mode, while each battery sub-stack is operational (i.e., prior to the output voltage discontinuity), the amp-hrs needed to power the operational mode are provided by both battery sub-stacks.

These graphs were created using an iterative test procedure using a battery pack 126 having the same construction as that used in the simulated life testing discussed above. Starting with new batteries, a 50 ohm resistor was placed across the terminals of the battery pack 126 for 40 minutes. The 50 ohm resistor was removed and the voltage output determined. Using the known voltage output, a resistor giving a load consistent with a current draw of 1 mA was connected across the battery pack 126 terminals, and the current from each battery sub-stack obtained. Then, a resistor giving a load consistent with 100 mA was connected across the battery terminals, and the current from each battery sub-stack obtained. Finally, the procedure was conducted with a resistor giving a load consistent with a 1.5 A draw. This iterative procedure was repeated some number of times. The average voltage output from the battery pack 126 over the test was 11V.

As shown in FIG. 6, when a very low current is drawn, current is drawn predominantly from one battery sub-stack. It should also be observed that there is a change over between sub-battery stacks. Initially, the battery sub-stack 404 is providing the bulk of the current and then there is a change over to battery sub-stack 402. This results due to the ever increasing voltage drop present in the failing battery sub-stack.

FIGS. 7 and 8 show that as current drawn from the battery pack 126 increases, current sharing between the battery sub-stacks becomes less disproportionate. At the highest of current draws there is only a minor difference between the proportions of the current load being satisfied by either battery sub-stack. Thus, where the current draws are low (i.e., low load), one battery sub-stack provides the capacity. But when, the current draws are high (i.e., high load), the current draw is allocated more equally.

As those skilled in AED design will appreciate, many AEDs are intended to meet a once in a life time need, but have many operational modes whether in storage or in use that use battery pack capacity at varying rates. For example, during storage, an AED continually performs scheduled self tests. These self tests vary in scope and duration. For example, a daily self test uses very little battery capacity, while weekly, monthly and quarterly self tests use ever increasing amounts. Generally, the increased amount of battery capacity used in various self tests results from degree the testing involves the shock circuit. In tests that are more frequent, the shock system may be not charged or only partially charged where in the less frequent tests it could fully, or almost be fully, charged.

For example, when stored and OFF with no self-testing occurring (e.g., the AED is merely reporting operational status using an active indicator), the load and associated current draw is in single digit milliamps, but relatively continuously. When OFF and conducting a daily self-test, the load is marginally higher having a current draw in the hundreds of milliamps (e.g., 100-200) for some short duration. However, when OFF and performing weekly, monthly, or quarterly self-tests, the load can be significant with the current draw (either battery limited or device limited) approaching several amps (e.g., 2 amps) for some number of seconds, becoming longer for the less frequent tests (e.g., 2 seconds weekly, 10 seconds quarterly). In the event of the AED is used in a rescue, the load and associated current draw is generally equivalent in amount and duration to that in longest self-test.

Referring to FIGS. 6-8 and assuming an AED is maintained for a random emergency, the AED will be predominately OFF with no-self-testing occurring, thus it will operate predominately using a single battery sub-stack. Even when OFF and conducting daily self-testing, one battery stack will be predominately used. However, during extremely high current draw events, such as during non-daily self-testing and rescues, both sub-stacks will more equally participate in the operation of the AED.

The above usage pattern of the battery pack 126 makes diodes preferred for the load allocator 407, as diodes have generally constant voltage drops over a wide current range. This diode characteristic maximizes battery pack 126 life by keeping the voltage drop associated with the load allocator 407 as small as possible under all potential AED uses, even during high current events. Schottky diodes, which are illustrated, are available with forward-voltage drops between approximately 0.15-0.45 volts. Other more conventional diodes, such as silicone diodes, could be used, but the available forward-voltage drops are between approximately 0.7-1.7 volts. Precise diode selection is a matter of design choice considering such factors as maximum current flow and maximum reverse voltage.

As discussed above, the significant drop, or discontinuity, in output voltage indicates a failure in battery sub-stack 404. As those skilled in the art will appreciate, the diodes used in the battery stack affect when the significant drop in output voltage of the battery pack 126 will occur. More specifically, the objective is to create a different voltage drop between the branches of the circuit containing the battery sub-stacks. The closer the created voltage drops are, the longer the time until the significant drop will occur, assuming two equal battery sub-stacks. As a result, less residual capacity will remain in the battery pack 126, or in the still functioning battery sub-stack. On the other hand, the greater the disparity in the voltage drops, the shorter the time until the significant drop and the greater the residual capacity in the battery pack 126 or the still functioning battery sub-stack.

It, therefore, should be appreciated that there is a tradeoff between the amount of residual capacity and the timing of the occurrence of the significant voltage drop. As the significant voltage drop is used to signal the need to replace the battery pack, this will establish the duration of the notice period before AED failure, and the time in which the battery must be replaced to avoid an out-of-service condition.

As addressed above, the voltage discontinuity can be used as a triggering event for the AED to notify a user of the need to replace the battery pack 126. For example, during a self-test, the self-test could determine the output voltage of the battery pack under a known load condition, such as a “battery test event.” Then based on a pre-determine threshold voltage, determine whether to alert the user to the need to replace the battery pack. The threshold voltage would be set between the output voltage before the discontinuity and the output voltage after the discontinuity.

In the alternative, self-tests that run frequently on the AED, such as periodically, would determine a change in output voltage of the battery pack 126 by comparing the ultimate output voltage with a previous output voltage. For example, a self-test is run in which an output voltage of the battery pack 126 is determine and then this ultimate output voltage is compared to the penultimate output voltage. The delta between the two, would be compared to a predetermine voltage delta and if equal to or greater than the predetermined voltage delta, the programming would trigger some type of user alert, such as through the ASI. It would also be possible for programming to compare some number of prior output voltages, such as five prior output voltages be they the last five or say five of the last 10. For those skilled in the art of programming AEDS, the programming required is straight forward based on the description of the requirements provided.

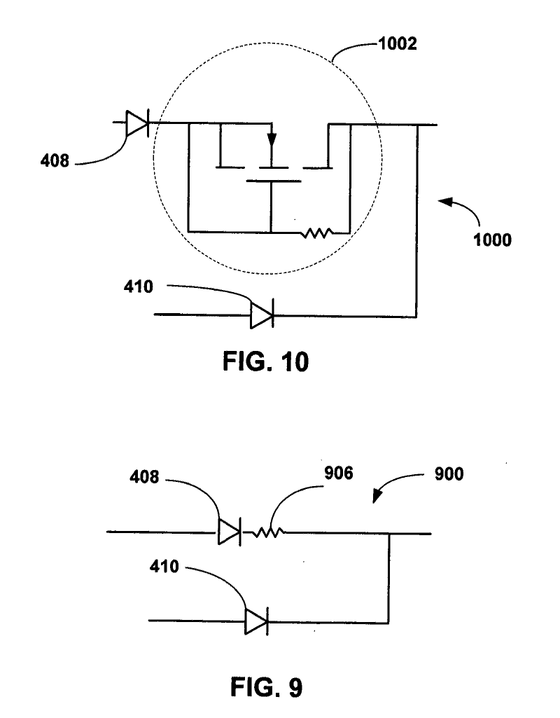

FIG. 9 is another embodiment of the load allocator 407, referred to by reference no. 900 with common components having the same reference numbers. In this embodiment, one of the series diodes is replaced with a resistor 906.

FIG. 10 is another embodiment of the load allocator 407, referred to by reference no. 1000 with common components having the same reference numbers. In this embodiment, one of the series diodes is replaced with a MOSFET 1002. The MOSFET is configured as a diode, and provides a low voltage drop. A suitable MOSFET is a LINEAR TECH LTC4358. It should be appreciated, that the diode 408 could be integrated into the MOSFET.

In addition, other diode configurations could be used. More specifically, a single Schottky diode could be used on one branch and a single silicone diode on the other. As a result, each branch could only have one diode instead of one branch having two. As applied to the embodiment depicted in FIG. 4, the diode 408 and diode 412 would be combined into one diode, where the one diode would have a voltage drop greater than that of diode 410.

Alternative embodiments of the invention will become apparent to one of ordinary skill in the art to which the present invention pertains without departing from its spirit and scope. Thus, although this invention has been described in exemplary form with a certain degree of particularity, it should be understood that the present disclosure has been made only by way of example and that numerous changes in the details of the construction and the combination and arrangement of parts or steps may be resorted to without departing from the spirit or scope of the invention. Accordingly, the scope of the present invention is defined by the appended claims rather than the foregoing description.

What is claimed is:

1. A battery pack for a device comprising:
two battery sub-stacks, each battery sub-stack initially has a capacity sufficient of power the device, and
a load allocator, electrically connecting the two battery sub-stacks in parallel, and misbalancing the capacity draw between the two battery sub-stacks,
whereby one battery sub-stack will fail before the other.

2. The battery pack of claim 1 wherein the load allocator is a passive device.

3. The battery pack of claim 1 wherein the load allocator includes two diodes in series with one battery sub-stack and one diode in series with the other battery sub-stack, the two diodes and one diode being in parallel.

4. A method of notifying a user to replace a battery pack in a device comprising the steps of:
obtaining a device powered by a battery pack having a capacity and having operational modes, some operational modes having a different battery pack capacity usage, wherein
the battery pack includes two battery sub-stacks,
the battery sub-stacks being connected in parallel,
means for misbalancing the capacity draw between the connected in parallel battery sub-stacks, and
each connected in parallel battery sub-stack initially has the capacity to operate the device in all operational modes,
self-test programming running on the device to evaluate the operational status of the battery pack based on output voltage and determine a failure in a battery sub-stack based on a discontinuity in the output voltage, and
an active status indicator autonomously operated by the self-test programming for outputting to a user the status of the battery pack as determined by the self-test;
frequently testing the battery pack to determine a discontinuity in the output voltage;
upon determining a discontinuity in the output voltage, the battery pack continuing to be able to operate the device in all operational modes for some period of time, and notifying the user during the some period of time using an active status indicator to replace the battery pack.

5. An automated external defibrillator comprising:
programmable circuitry having programming running thereon capable of analyzing a heart rhythm to determine if a defibrillation shock is appropriate
circuitry operated by the programmable circuitry capable of delivering a shock to a person, if appropriate,
a battery pack powering the circuitry and programmable circuitry, the battery pack including
two battery sub-stacks electrically connected in parallel, the connection in parallel defining two branches, wherein each battery sub-stack initially has a capacity sufficient of power the AED, and
a means for misbalancing the capacity draw between the two battery sub-stacks.

6. The automated external defibrillator of claim 5 wherein the programmable circuitry can autonomously direct the delivery of a shock.

7. The automated external defibrillator of claim 5 wherein the circuitry operated by the programmable circuitry includes a manual switch that must change states to deliver a shock.

8. A method of notifying a user to replace a battery pack in a device comprising the steps of:
obtaining a device powered by a battery pack having a capacity and having operational modes, some operational modes having a different battery pack capacity usage, wherein
the battery pack includes two battery sub-stacks,
the battery sub-stacks being connected in parallel,
means for misbalancing the capacity draw between the connected in parallel battery sub-stacks, and
each connected in parallel battery sub-stack initially has the capacity to operate the device in all operational modes,
self-test programming running on the device to evaluate the operational status of the battery pack based on output voltage and determine a failure in a battery sub-stack based on a pre-determined threshold voltage, and
an active status indicator autonomously operated by the self-test programming for outputting to a user the status of the battery pack as determined by the self-test;
frequently testing the battery pack to determine a discontinuity in the output voltage;
upon determining a discontinuity in the output voltage, the battery pack continuing to be able to operate the device in all operational modes for some period of time, and notifying the user during the some period of time using an active status indicator to replace the battery pack.
